(19) United States
(12) Patent Application Publication
(10) Pub. No.: US 2002/0072862 A1
(43) Pub. Date: Jun. 13, 2002

(54) Creation of a unique sequence file

(76) Inventor: Christophe Person, The Woodlands, TX (US)

Correspondence Address: C. Steven McDaniel, Esq., McDaniel & Associates, P.C., P.O. Box 2244, Austin, TX, 78768-2244, US

(21) Appl. No.: 09/933,056

(22) Filed: Aug. 20, 2001

Related U.S. Application Data
(60) Provisional application No. 60/227,100, filed on Aug. 22, 2000.

(51) Int. Cl. C12Q 1/68; G06F 19/00; G01N 33/48; G01N 33/50

(52) U.S. Cl. 702/20; 435/6

(57) ABSTRACT

This disclosure teaches a computerized method for finding new Unique Sequences and sequence fragments via a Region Definition Procedure. New Unique Sequences can be recognized when an unknown Query Sequence is compared and aligned with a plurality of previously stored sequence fragments. Using a Region Definition Procedure, each of the aligned sequences has a beginning and an end point that defines a Region that is compared directly with the Query Sequence during the alignment process. New Unique Sequences within the Query Sequence are identified and stored in a UNIQUE FILE for future use in identifying Unique Sequences for further investigation.

### FIELD OF THE INVENTION

The present invention relates to a system for using a computerized Region Definition Procedure in the creation of a Unique Sequence file.

### BACKGROUND OF THE DISCLOSURE

Nucleic acids (DNA and RNA) carry within their structure the hereditary information and are therefore the prime molecules of life. Nucleic acids are found in all living organisms including bacteria, fungi, viruses, plants and animals and they make up the genes within the cell. It is estimated that there are over 100,000 genes within the genome of the human cell. It is of interest to determine the relative abundance of certain nucleic acids sequences in different cells, tissues and organisms over time under various conditions, treatments and regimes. The nucleic acids code for the amino acids, which are the molecular building blocks of proteins. Proteins are found within the cells of an organism and function to keep the cells alive and responding to it's environment.

Informatics is the study and application of computer and statistical techniques to the management of information. Bioinformatics and computation in biological research have changed dramatically in the last decade. Increasingly, molecular biology is shifting from the laboratory bench to the computer desktop. Today's researchers require advanced quantitative analyses, database comparisons, and computational algorithms to explore the relationships between sequence and phenotype. New observational and data collection techniques have expanded the capabilities of biological research and are changing the scale and complexity of biological questions that can be productively posed.

The structures of coding and non-coding DNA sequences and amino acid sequences of many organisms have been analyzed, and information concerning those sequences has been recorded in databases accessible via the World Wide Web for common use. Biomedical researchers can gain access to such public domain databases and utilize this information in their own research. Such databases include, for example, GenBank in the U.S., EMBL in Europe, DDBJ at National Gene Institute of Japan, and so on. Genetic information for a number of organisms has also been catalogued in computer databases. For example, genetic databases for organisms such as Escherichia coli, Caenorhabditis elegans, Arabidopsis thaliana, and Homo sapien sapien, are publicly available. At present, however, complete sequence data is available for relatively few species and the ability to manipulate sequence data within and between species and databases is limited.

The new wealth of biological data generated by ongoing genome projects is being used by biologists in combination with newly developed tools for database analysis to ask many questions from molecular interactions to relationships among organisms. Bioinformatics, is contributing to the usefulness of the information generated by the genome projects with the development of methods to search databases quickly, to analyze nucleic acid sequence information, and to predict protein sequence, structure and correlate gene function information from DNA sequence data. Comparisons of multiple sequences can reveal gene functions that are not evident in any single sequence. Web-based searches of several collections of amino acid sequence motifs can elucidate particular structural or functional elements.

Biological sequence databases, though, contain many repeated and redundant sequences or sequence fragments. These repeated and redundant sequences or sequence fragments have been deposited in the public sequence repository databases as many as three or more times. Sequences may be deposited redundantly because often researchers from different laboratories determine the sequences of the same gene or chromosome segment from the same or closely related species. Some identical or closely related sequences have been deposited approximately 10³ times in the biological sequence databases. Repeated sequences appear naturally in the DNA/RNA and are deposited as part of a whole sequence or fragment. In addition, a variety of experimental protocols contribute to the increase of contamination sequences deposited in databases. Because of such contamination, some chimeric sequences produced from different genes of different species (yeast, bacteria, etc.) may be present.

With the thousands of sequences or sequence fragments being added to the databases everyday there is a need for a faster, more efficient means of searching these sequences for Unique Sequences that have never been identified before. Redundancies in the currently available DNA/RNA databases render the systematic analysis of similarity or homology between DNA/RNA sequences impractical both in terms of computation and time. The conventional bioinformatic algorithms available do not address this problem.

### SUMMARY OF THE INVENTION

The disclosure teaches a method for identifying Unique Sequences within Redundant Sequence Database Files (RED FILES) via a Region Definition and Unique Sequence Identification procedure. Sequences from the RED FILES can be searched and rendered more useful by first identifying sequence regions that define Unique Sequences or fragments of sequences within the Query. Subsequently, such identified Unique Sequences or sequence fragments can be stored in a separate Unique Sequence Database File (UNIQUE FILE).

One aspect of the invention is a database of unique nucleotide sequences comprising nucleotide sequences greater the 100 nucleotides in length.

Another aspect of the invention is a method for generating a database of sequences that are greater than or equal to 100 nucleotides in length, wherein each sequence is entered into the database only one time. The method of generating a Unique Sequence database has the following steps: a) selecting a query sequence from a redundant database; b) masking said query sequence with known repeat sequences; c) comparing said masked query sequence with identified unique sequences; d) identifying a unique portion of the query sequence that does not have a similar sequence in any of the identified unique sequences; and e) adding the unique portion of the query sequence to a unique database.

Yet another aspect of the invention is a method for identifying unique nucleotide sequences comprising: a) selecting a query sequence from a redundant database file; b) comparing the query sequence with a repeat database file and a unique database file; c) analyzing the results of the comparison of the query sequence with the repeat database file and the unique database file to determine if there is one or more nucleotide sequences within the repeat database file and the unique database file that match a nucleotide sequence within the query sequence; and d) identifying any unique nucleotide sequences within the query sequences that do not match any nucleotide sequence within the repeat database file and the unique database file.

The foregoing has outlined rather broadly the features and advantages of the present invention in order that the detailed description of the invention that follows may be better understood. Additional features and advantages of the invention will be described hereinafter which form the subject of the claims of the invention.

### BRIEF DESCRIPTION OF THE DRAWINGS

The novel features which are believed to be characteristic of the invention will be better understood from the following detailed description, in conjunction with the accompanying drawings.

FIG. 1A. Illustrates a preferred ordering of subsets of Redundant Sequence Database Files (RED FILES).

FIG. 1B. Illustrates a flow diagram of the key steps employed in identifying Unique Sequence Regions for a Unique Sequence Database File (UNIQUE FILE) using the Query Sequences chosen from RED FILE subsets.

FIG. 2. Illustrates a flow diagram that presents key steps employed in identifying Unique Sequences for a Unique Sequence Database File using the Independently Derived (ID) Sequences as the Query Sequence.

FIG. 3. Illustrates a pairwise sequence alignment with gaps in the sequences, where the bases of Qᵢleft and. Qᵢright align exactly with Hᵢleft and Hᵢright from the Query/Hit pairwise alignment fragmentᵢ.

FIG. 4A. Illustrates three examples of pairwise alignments where the Hit sequence fragments are lined up in relationship to the original Query Sequence.

FIG. 4B. Illustrates how Boundary Regions are defined using a graphical local multiple sequence alignment output with three Hit Sequences.

FIG. 5A. Illustrates three examples of pairwise alignments.

FIG. 5B. Illustrates how Boundary Regions are defined using a local graphical multiple sequence alignment output with three Hit Sequences.

FIG. 6A. Illustrates the multiple Hits of sequence or sequence fragments per Region per search that are typically obtained using the RED FILE

FIG. 6B. Illustrates the single Hit of sequence or sequence fragments per Region per search that is obtained using the UNIQUE FILE.

### DETAILED DESCRIPTION OF PREFERRED EMBODIMENTS

This disclosure teaches a computerized method of a Region Definition Procedure that increases the efficiency of standard bioinformatics tools and databases. This procedure is designed to enhance the specialized needs of a high-throughput genomics-computing environment by identifying Unique Sequences and storing them in a Unique Sequence Data File (UNIQUE FILE).

Existing databases contain repeated sequence fragments that have been inherited during evolution by many different unrelated genes. These repeated sequences create a special problem when searching the public domain databases for Unique Sequences. If a given Query Region corresponds to redundant or repeated sequences or sequence fragments, the large number of resulting matches will often obscure interesting relationships to other non-related or less related genes.

A fast and efficient means of building a UNIQUE FILE containing only one copy of known sequence fragments is disclosed. This computerized method is used to identify Unique Sequences or sequence fragments from one or more Query Regions that have never been placed in the UNIQUE FILE. The UNIQUE FILE contains only one copy of sequences corresponding to Regions from Queries that have been recognized through homologous hits via pairwise sequence alignments. Sequences or sequence fragments that have an equivalent sequence previously placed in the UNIQUE FILE and/or the Repeat File (REP FILE) are ignored and not added to the UNIQUE FILE again. A sequence or sequence fragment that has previously been added to the UNIQUE FILE can be detected if a Region on the Query is recognized when a matching sequence from the UNIQUE FILE results in a Hit. If a Hit occurs the corresponding Region for that Query Sequence is ignored and not added to the UNIQUE FILE.

1. Relevant Terminology

There is some ambiguity in the scientific literature as to the relevant nomenclature, so it is important to define some specific terms within this disclosure. The following bioinformatics terms are used to define concepts throughout the specification. The descriptions are provided to assist in understanding the specification, but are not meant to limit the scope of the invention.

A Repeat Sequence Database File (REP FILE) is composed of sequence domains or sequence fragments that are known to be present in multiple copies in a single genome, etc. (e.g., Alu sequences).

A Unique Sequence Database (UNIQUE FILE) is composed of sequences or sequence fragments that are known to be representing a Unique Sequence Region never identified from a Query Sequence before within the UNIQUE FILE. These Unique Sequence Regions or sequence fragments are identified using the Boundary Definition and Unique Sequence Identification Procedure.

Public Domain Sequence Databases are databases available for use by the public. Typically, such databases are maintained by an entity that is different from the entity creating and maintaining the UNIQUE FILE and REP FILE. In the context of this invention, the public domain databases are used primarily to obtain information about the Query Sequences obtained from other sequencing laboratories around the world. Examples of such Public Domain Databases include the GenBank and dbEST databases maintained by the National Center for Biotechnology Information (NCBI), TIGR database maintained by The Institute of Genomic Research and SwissProt maintained by ExPasy.

An Independent Sequence Database is a database that contains Independently Derived Sequence data obtained and processed by the database developer.

Independently Derived Query Sequence (ID Query) is a sequence that has been generated within the in-house sequencing laboratory of the database developer.

Redundant Files (RED FILES) include public domain sequence databases and Independent Databases that contain redundant sequences. Query Sequences are selected from the RED FILES and generally contain redundant sequences. Redundant sequences or sequence fragments have been deposited in the sequence repository two or more times. Sequences may be deposited multiple times because researchers from different laboratories determine the sequences of the same gene or chromosome segment from the same or closely related species or because the sequence is a commonly repeated sequence domain within a gene. Some identical or closely related sequences have been deposited approximately 10³ times in the public domain sequence databases, generating redundancies that are costly in terms of processing and analysis.

Target database(s) are databases of pre-existing sequences to which the Query Sequence will be compared to find the most similar matches (example: UNIQUE and REP FILES).

Database Search Algorithms are mathematical means of identifying similar sequence Regions within a Query Sequence when compared to database sequences. BLAST, FASTA, Smith-Waterman are common examples of database search algorithms that can produce a list of pairwise alignments between a Query Sequence and all matching (Hit) sequences in searchable sequence databases.

A Cluster is a group of sequences related to one another by sequence similarity. Clusters are generally formed based upon a specified degree of homology similarity and overlap.

An Algorithm is a mechanical or recursive computational procedure for solving a problem.

A Multiple Sequence Alignment (MSA) is a group of three or more sequences aligned to maximize the registry of identical residues. Global MSA are sequence alignments that require the participation of all sequence residues. For the purpose of this disclosure Local MSA will be used that does not require the participation of all sequence residues in the alignment. MSA is the process of aligning several related sequences, showing the conserved and non-conserved residues across all of the sequences simultaneously. These conserved/non-conserved residues form a pattern that can often be used to retrieve sequences that are distantly related to the original group of sequences. These distant relatives are extremely helpful in understanding the role that the group of sequences plays in the process of life. This can be the alignment of like nucleic acid residues of several genes or the amino acids of a number of protein sequences. The final product of a MSA may contain a gap character, “-”, which is used as a spacer so that each sequence has the same number of residues plus gaps in the alignment. A MSA shows the residue juxtaposition across the entire set of sequences; thus showing the conserved and non-conserved residues across all of the sequences simultaneously.

A Scoring Matrix is a table of values used to evaluate the alignment of any two given residues in a sequence comparison. For protein sequences there are two main families of scoring matrices: PAM and BLOSUM.

FASTAlign is Lexicon Genetics' software program for the rapid construction of multiple sequence alignments from nucleotide and protein sequences. FASTAlign is a multiple sequence alignment algorithm similar to NCBI's N-align.

BLAST (Basic Local Alignment Search Tool) is a set of database search programs designed to examine sequence databases. BLAST uses a heuristic algorithm which seeks local as opposed to global alignments and is therefore able to detect pairwise relationships among sequence fragments which share only isolated regions of similarity (Altschul et al., 1990).

FASTA is a set of sequence comparison programs designed to perform rapid pairwise sequence comparisons. Professor William Pearson of the University of Virginia Department of Biochemistry wrote FASTA (Pearson, William, 1990). The program uses the rapid sequence algorithm described by Lipman and Pearson (1988) and the Smith-Waterman sequence alignment protocol.

The Smith-Waterman Algorithm is a modification of the global alignment method that efficiently identifies the highest scoring sub-region shared by two sequences (Smith and Waterman, 1981, Waterman, M. S., 1989 and Waterman, M. S., 1995). Often homologous sequences only share similarity in a small sub-region. Global alignments may fail to include such Regions of relatedness in an end-to-end optimal alignment.

An Expectation Threshold (ET) is the length of a sequence alignment determined to be necessary to distinguish between evolutionary relationships and chance sequence similarity. The ET is calculated using normalized probability scores. The ET selected will vary based on the amount of error one is willing to accept. For example, an ET of 8 nucleotides can be accepted if one is willing to accept an 8-10% error. If one is only willing to accept a small percentage error, then the ET selected must be a longer nucleotide sequence. Preferably, a minimum ET of 100 nucleotides is selected for determining if a portion of a Query Sequence is a Unique Sequence. However, where a Hit contains a relatively small area having no matching nucleotides in the Query Sequence, an ET of about 30 nucleotides may be selected.

N-Align is a program that NCBI uses to recast the standard bioinformatic database output. The Query/Hit Sequence pairs, identified from database searches, are aligned to the full Query Sequence. This alignment format exists in graphical and text renditions in the NCBI search outputs.

A Sequence Database Search Output consists of a collection of one or more identified pairwise alignments in a Query/Hit Sequence pair that exceeds a designated expectation threshold (ET).

A Pairwise Alignment is an alignment of a part or the whole of two sequences.

Pairwise alignment software is a program used to recast the standard bioinformatics database output. The Query/Hit Sequence pairs, identified from database searches, are aligned to the full Query Sequence. This alignment format exists in graphical and text renditions in many public search outputs.

A Sequence Alignment is a comparison between two or more sequences that attempt to bring into register identical or similar residues held in common by the sequences. It may be necessary to introduce gaps in one sequence relative to another to maximize the number of identical or similar residues in the alignment.

A Hit is when two or more sequences are brought together into register with identical or similar residues that are held in common by those sequences in a pairwise alignment.

The following definitions are used to define molecular biology terms throughout the specification. These definitions are provided to assist in understanding the specification, but are not meant to limit the scope of the invention.

A contig is a group of overlapping DNA segments.

A contig map is a chromosome map showing the locations of those Regions of a chromosome where contiguous DNA segments overlap. Contig maps are important because they provide the ability to study a complete, and often large segment of the genome by examining a series of overlapping clones which then provide an unbroken succession of information about that region.

A Consensus sequence is a nucleotide sequence constructed as an idealized sequence in which each nucleotide position represents that base most often found at that position when many related nucleotide sequences are compared. Variations of mismatch nucleotides compared to consensus sequences may characterize single nucleotide polymorphisms (SNPs) representing the diversity or polymorphism of a particular gene in the population or species.

A Concatamer is a global consensus sequence created by joining end to end overlapping sequence fragments and merging areas of the overlap.

A Gene is the functional and physical unit of heredity passed from parent to offspring. In this disclosure the term gene is intended to mean a sequence of bases of DNA or mRNA bases containing the information to code for a sequence of amino acids that make up a protein.

2. Sequence Query Acquisition and Building a Unique File.

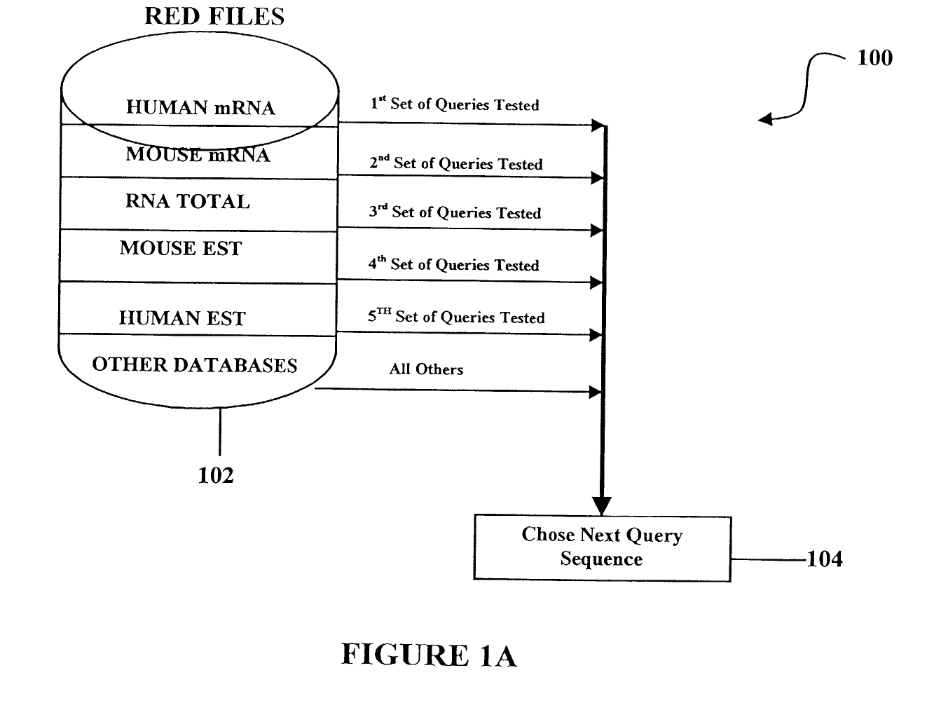

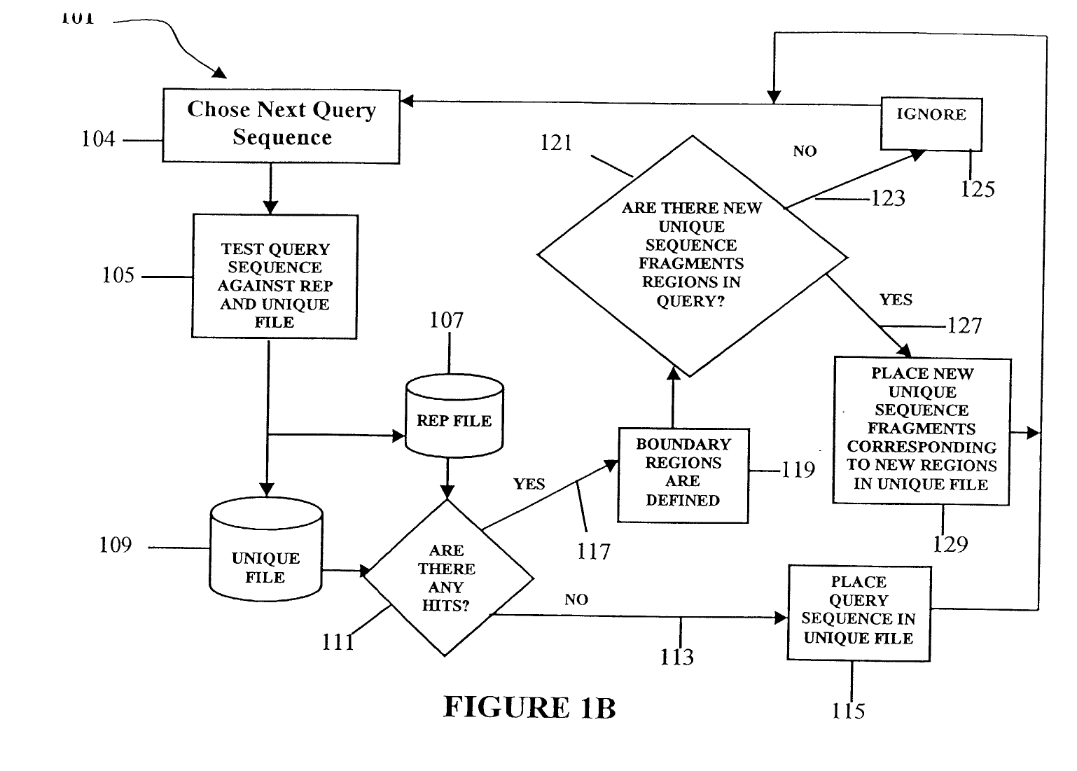

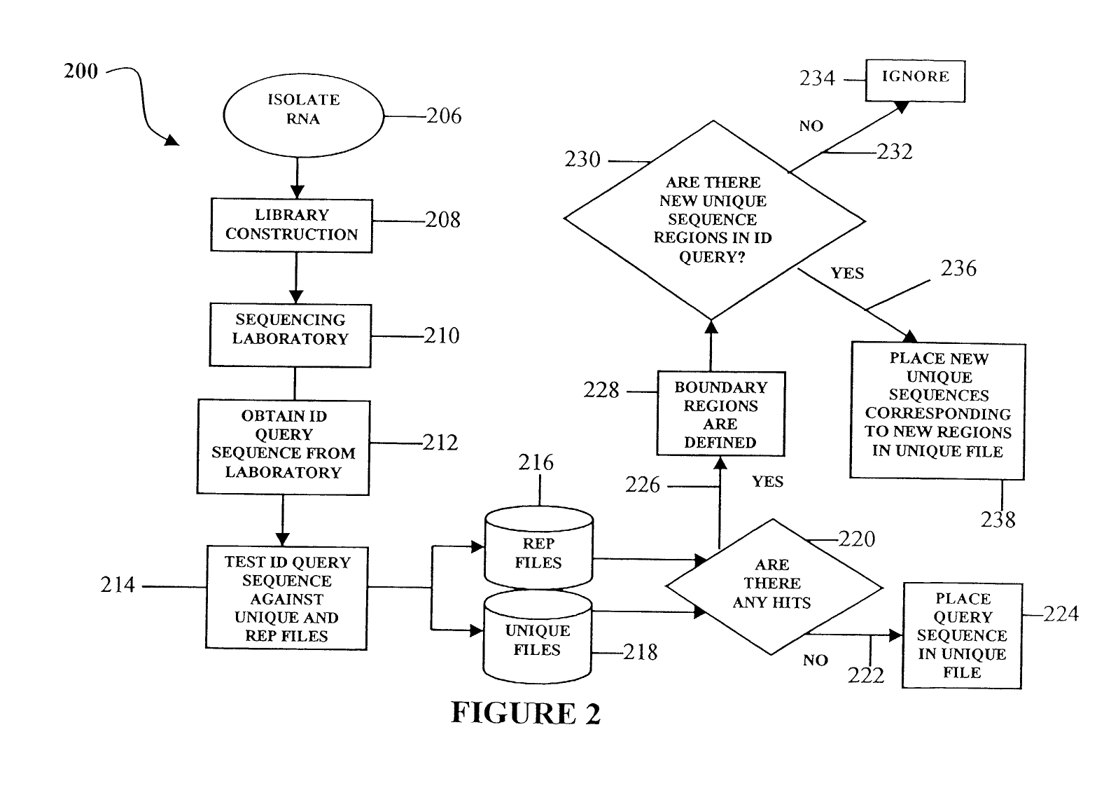

FIGS. 1A, 1B and 2 illustrate a preferred embodiment of a computerized method of identifying Unique Sequences within the Redundant Sequence Database Files (RED FILES) via a Boundary Region Definition and Unique Sequence identification procedure and placing them into a UNIQUE FILE. A more detailed discussion of the steps in this process is described below.

Sequences from subsets of the RED FILES can be searched and rendered more useful by also identifying repeated sequences within them. Subsequently, identified repeated sequences are stored in a separate Sequence Repeat Database File (REP FILE) for future identification.

As shown in FIG. 1A a Query Sequence 104 is selected for a Unique Sequence search from an ordered subset of RED FILES 102. For access to the most useful data available, this subset of the RED FILES has been ordered by species and by annotation richness.

The first set of Query Sequences is often selected from the Human mRNA database files in the RED FILES 102. The Human database subset is the most relevant species for medical research and is typically the first database to be searched for Unique Sequences. Furthermore, the Human mRNA databases have very rich or excellent annotations. All annotations associated with the selected Query Sequences will be maintained and stored with the Query Sequence or any subsequently identified fragment thereof. However, depending on the Query sequence, it may be relevant to use other species sequences. In the following paragraphs Human can be substituted with any other species, depending on the intent and goals of the user.

Mouse mRNA database files, which are very large database files with very good annotations, is generally searched for Unique Sequences after the Human mRNA subset has been searched. Other database subsets, such as the total RNA, Mouse EST and Human EST, are preferably searched in the order of the richness of their annotations and future usefulness in correlating gene function and location information from genomic DNA sequence data. However, if the investigator were interested specifically in the mouse database files, Queries from the mouse RNA database files would be selected first.

As shown in FIG. 1B the selected Query Sequence 104 will be tested 105 against the Repeat Sequence Database (REP FILE) 107 and the Unique Sequence Database (UNIQUE FILE) 109. The REP FILE is composed of sequences and fragments that are not unique and are known to be present in multiple copies in a single genome (e.g., Alu sequences, E. Coli sequences, blue script sequences, etc.). These sequences may be present in the selected Query Sequence 104 and must be identified and masked so that they are not considered Unique Sequences. The UNIQUE FILE 109 is composed of sequences or sequence fragments that are known to be unique and have never been identified before within the UNIQUE FILE 109 and the REP FILE.

The analysis systems, represented by step 105 in process flow 101 (FIG. 1B) may use typical programs, such as the Smith-Waterman algorithm (Smith and Waterman, 1981, Waterman, M. S., 1989 and Waterman, M. S., 1995), the BLAST programs (Altschul et al., 1990), or the FASTA program (Pearson, William, 1990, Lipman and Pearson, 1988), or any pairwise sequence alignment program or method to test the Query Sequence.

These programs use rapid sequence alignment algorithms that produce a list of pairwise alignments. A parsing program scans the pairwise alignments produced and accumulates them in a buffer. These pairwise alignments are reduced and contigs are created which are then processed back through the sequence alignment algorithm as a new Query Sequence. This alignment and parsing continues until the Query Sequence alignment process identifies all known-matching sequences in the target databases. Scoring Matrix Programs such as PAM (M. O. Dayhoff, 1978) or the BLOSUM families (Henikoff and Henikoff, 1992) are used to evaluate the matches of the alignment and Expect Values of Altschul (Altschul et al., 1997) is the method of ranking the scores of the matches. Due to sequence polymorphism, and in the context of several million analyses, the validity of the matches may be reevaluated by other methods in the context of gene specificity. FASTAlign then recasts the compiled text listings of these pairwise alignments into a graphical rendition.

After testing the Query Sequence 104 against the REP FILE 107 and the UNIQUE FILE 109 the question is asked, “Are there any Hits?” 111. If there are no Hits on the Query Sequence 104 with a sequence or sequence fragments that were previously stored in the UNIQUE FILE 109 or in the REP FILE 107, the answer is “NO” 113. The whole Query Sequence 104 is then considered a new Unique Sequence and is placed in toto into the UNIQUE FILE 115 and a new Query Sequence is obtained.

If one or more Hits do occur on the Query Sequence 104 after testing against the REP FILE 107 and the UNIQUE FILE 109 then the answer is “YES” 117 and Boundary Regions are defined 119 on the local multiple sequence alignments.

Boundary Regions (as described below in Example 1) are defined 119 using the local multiple sequence alignments created during the testing phase 105. Unique Sequences or sequence fragments are identified using the Boundary Region Definition and the Unique Sequence Identification Procedures. In step 121 the question is asked, “Are there new Unique Sequence fragment regions?” If there is a sequence fragment in a region that meets the pre-set conditions with no overlapping Hit fragment the answer is YES. Pre-set conditions are requirements that must be met by a region to be considered, such as, minimum length, percent quality of this Query region sequence, etc. A “YES” answer 127 will place these Unique Sequences in the UNIQUE FILE 129. A “NO” answer 123 signals that there is no new Unique Sequence or sequence fragments. The negative result is ignored 125 and a new Query Sequence is chosen.

3. Obtaining and Testing Independently Derived Sequence Queries.

Independently Derived Sequence Queries (ID Queries) may be obtained by various RNA isolation, reverse transcription and sequencing procedures known to those of skill in the art. In one example of such a procedure, total RNA from a particular human tissue culture line is isolated and reverse transcribed, purified, and the cDNA is cloned into suitable vectors for amplification. The vectors are then transformed into E. coli bacterial cells and grown overnight. Thereafter, multiple colonies, each representing a clone of a particular mRNA sequence of the organism, may be picked and used to create a cDNA library of clones. A selected colony's plasmid cDNA may then be isolated for sequencing. In the process flow of FIG. 2, the process begins at 206 when the total RNA is isolated and the library is constructed by step 208.

As represented by step 210, sequencing templates for a clone's cDNA are then prepared and sequencing reads are performed. Each cDNA sequence fragment is then specifically identified with an accession number.

The Independently Derived sequences or the ID Query Sequences, as they are called from this point forward are obtained from the sequencing laboratory in step 212. In process step 214, the ID Query Sequence is tested against 214 the REP FILES 216 which are composed of sequences and fragments that are not unique and are known to be present in multiple copies in a single genome and the UNIQUE FILE 218 which is composed of sequences or sequence fragments that are known to be unique.

The testing step 214 of the ID Query Sequence is performed using typical programs such as the Smith-Waterman algorithm (Smith and Waterman, 1981, Waterman, M. S., 1989 and Waterman, M. S., 1995), the BLAST programs (Altschul et al., 1990), or the FASTA program (Pearson, William, 1990, Lipman and Pearson, 1988), or any pairwise sequence alignment program or method to test the ID Query Sequence.

Using the local multiple sequence alignments generated during the testing 214 of the Query Sequence against the UNIQUE and the REP FILES the question is asked, “Are there any HITS?” 220. If there are not any Hits on the ID Query Sequence 212 with a sequence or sequence fragments that was previously stored in the UNIQUE FILE 218 or the REP FILE 216, the answer is “NO” 222. The whole ID Query Sequence 212 is then considered a Unique Sequence and is placed in toto into the UNIQUE FILE 224. If one or more Hits do occur on the ID Query Sequence 212 after testing against the REP FILE 216 and the UNIQUE FILE 218 then the answer is “YES” 226. and Boundary Regions are defined 228 on the local multiple sequence alignment produced during the testing phase 214.

Boundary Regions (as described below in Example 1) are then defined 228. Unique Sequences or sequence fragments are identified using the Boundary Definition and the Unique Sequence Identification Procedure. The gap scoring strategy tends to analyze a fragment's score as the gap extends. For this reason smaller fragments tend to score better than their longer gapped counterpart fragment. The question 230 is then asked, “Are there new Unique Sequence fragment regions?” If there is a sequence fragment in a region that meets the pre-set conditions with no overlapping Hit fragment the answer is YES. Pre-set conditions are requirements that must be met for a region to be considered, such as, minimum length, percent quality of this Query region sequence, etc. A “YES” answer 236 will place the Unique Sequence or sequence fragment in the UNIQUE FILE 238. A “NO” answer 232 signals that there is no new Unique Sequence on the Query Sequence. The negative result is then ignored 234.

### EXAMPLE 1

Region Definition Procedure

A. Comparison of Query Sequence with Target Database

A Query Sequence is compared with sequences in a Target database such as the REP and the UNIQUE FILES. Regions are defined based upon the relative position of the endpoints of the similar database sequence or Hit Sequence to the Query Sequence. Each sequence or sequence fragment in the target database that matches any or part of the Query Sequence is analyzed separately.

B. Identification of Endpoints on the Query Sequence

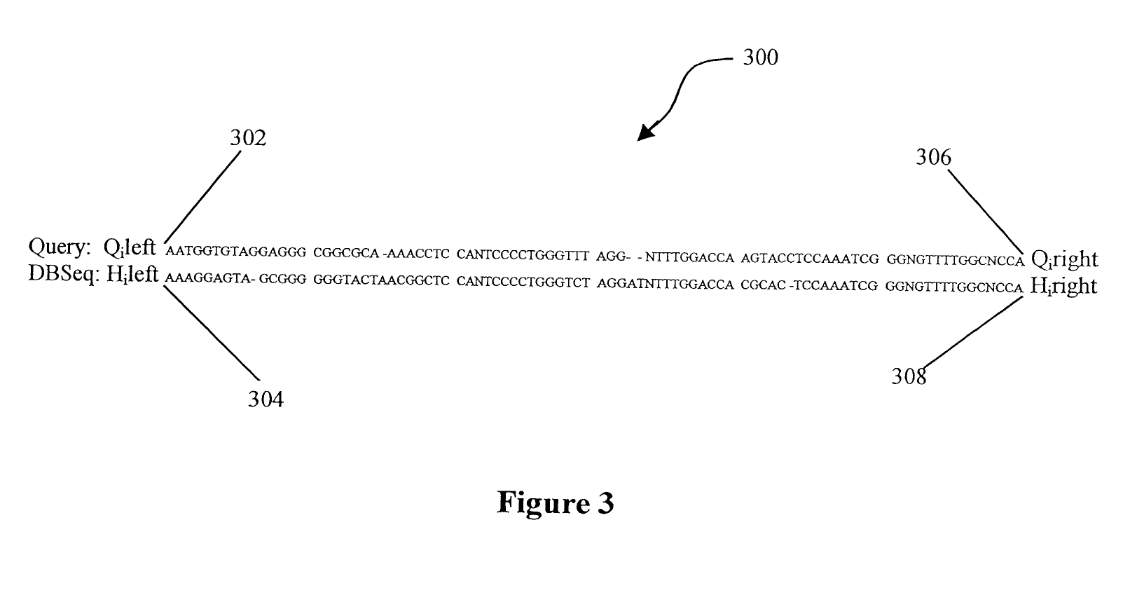

As illustrated in FIG. 3, the endpoints of the Query Sequence are defined as Qᵢleft 302, the left most absolute position of the Query Sequence or the left endpoint of the Query Sequence, and Qᵢright 306, the right most absolute position of the Query Sequence or the right endpoint of the Query Sequence. When a similar database sequence in the Target database is identified that matches a part or all of the Query Sequence it is then aligned with the part of the Query Sequence that it is similar to. For example, in FIG. 3 the Query Sequence and the similar database sequence (hereinafter referred to as a Hit) are almost identical. Thus, the left most absolute position of the Hit (Hᵢleft 304) matches the left most absolute position of the Query Sequence (Qᵢleft 302) where the nucleotide at 302 and the nucleotide at 304 are aligned exactly and represent the left most aligned nucleotide pair. Similarly, the right most absolute position of the Hit (Hᵢright 308) matches the right most absolute position of the Query Sequence (Qᵢright 306) where the nucleotide at 306 and the nucleotide at 308 are aligned exactly and represent the right most aligned nucleotide pair. The alignment of these two sequences represents one pairwise alignment.

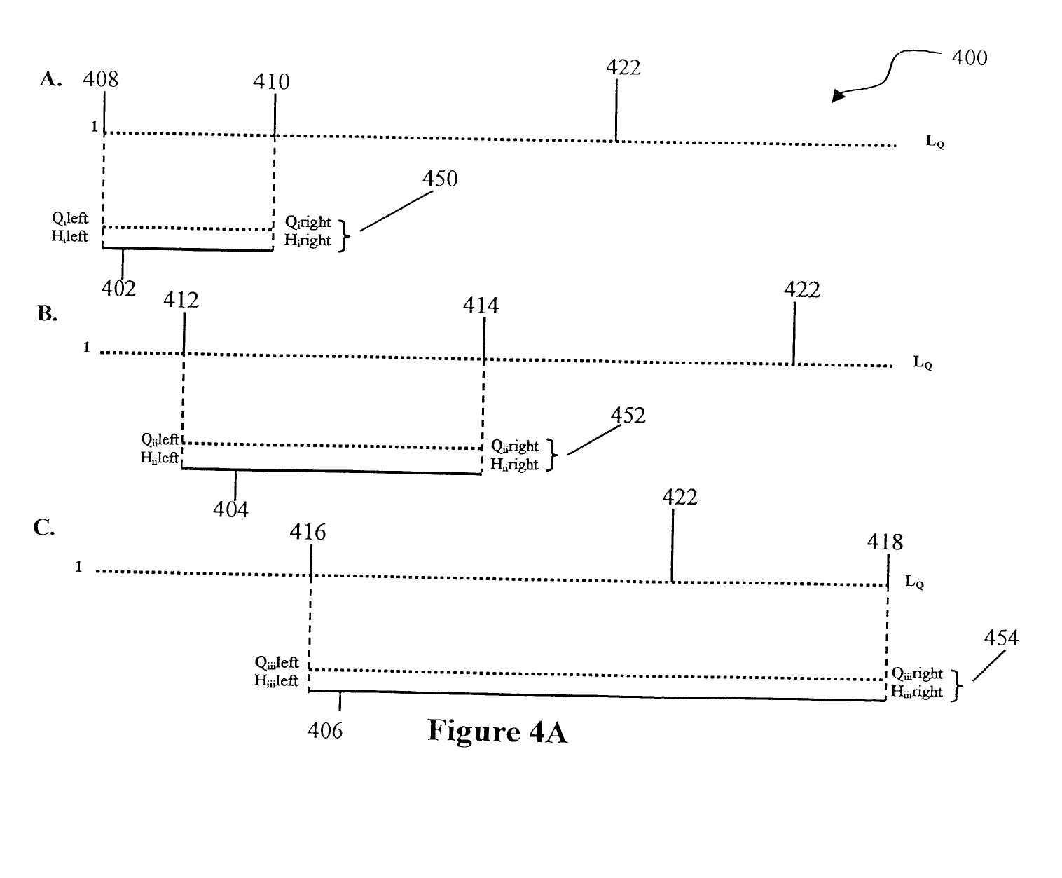

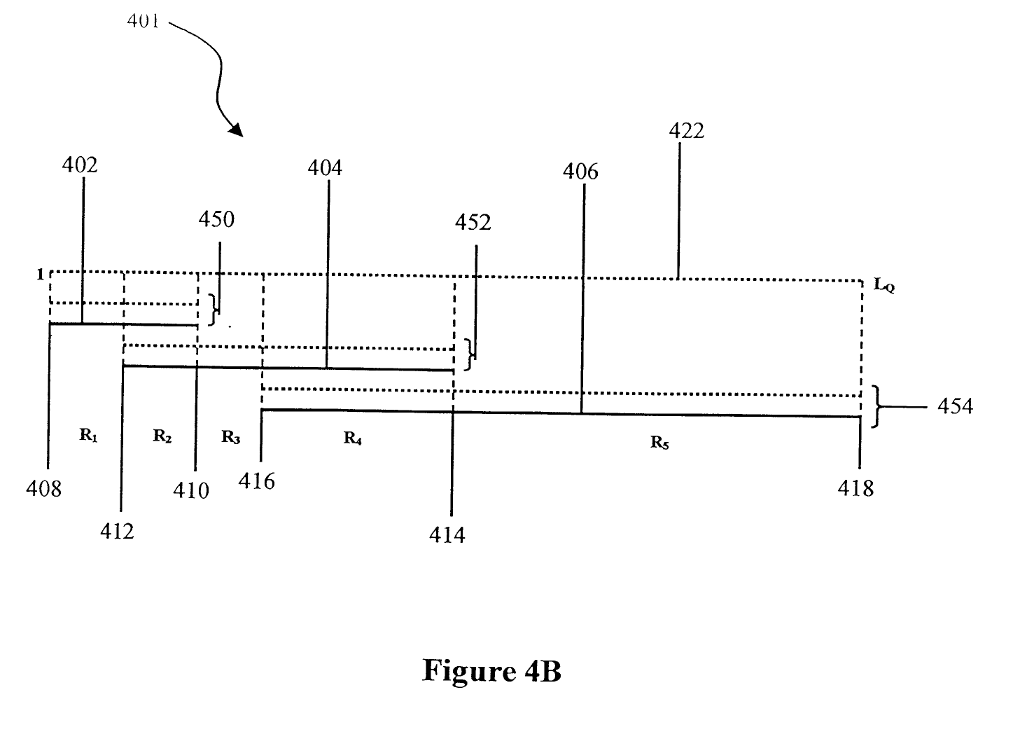

FIG. 4A illustrates the relative positional relationships between three Hit Sequences 402, 404, 406 and the Query Sequence 422. The first pairwise alignment 450 is composed of Hit Sequence 402 and a portion of the Query Sequence 422 between points 408 and 410. The second pairwise alignment 452 is composed of Hit Sequence 404 and a portion of the Query Sequence 422 between points 412 and 414. The third pairwise alignment 454 is composed of Hit Sequence 406 and a portion of the Query Sequence 422 between points 416 and 418. The Hit Sequences in the pairwise alignments are annotated with the nucleotide numbers from the Query Sequence 422 to which they correspond. For example, if the portion of the Query Sequence 422 between points 408 and 410 represents nucleotides 1 to 150, with the first nucleotide at left most end point being number 1, then the Hit Sequence 402 would be annotated to indicate that it matched the portion of the Query Sequence 422 between nucleotides 1 to 150.

C. Graphical Alignment of the Pairwise Alignments

Software programs such as NCBI's N-align or Lexicon Genetics' FASTAlign are used to recast the pairwise alignments into an ordered graphical format where each of the Hit Sequences are displayed below the entire Query Sequence aligned with the portion of the Query Sequence that it is similar to. FIG. 4B shows the graphical alignment of three Hit Sequences 402, 404 and 406 with their similar or homologous sequences aligning with matching areas on the Query Sequence 422.

D. Identifying Similar Sequence Regions

The graphical representation of the alignment of each Hit Sequence with their similar or homologous sequences on the Query Sequence 422 and overlap sequence fragments on any other contiguous Hit Sequence is used to determine the Boundary Regions in FIG. 4B. The endpoints of each Hit Sequence are visually connected to the Query Sequence 422. For example, Hit Sequence 402 left and right endpoints are connected to the Query Sequence 422 with dashed lines 408 and 410. The endpoints of Hit Sequence 404 have dashed lines 412 and 414 connecting it to the Query Sequence 422. Similarly, Hit Sequence 406 has dashed lines 416 and 418 connecting it to the Query Sequence 422.

Each of the lines that connect an endpoint of a Hit Sequence may intersect other Hit Sequences, if those Hit Sequences contain an overlapping sequence fragment to the initial Hit Sequence. For example, the dashed line 412 connecting the left endpoint of Hit Sequence 404 to the Query Sequence 422 intersects Hit sequence 402 and the dashed line 414 connecting the right endpoint of Hit Sequence 404 to the Query Sequence 422 intersects Hit Sequence 406. Dashed line 418 indicates the right endpoint of the Query Sequence 422 and the right endpoint of Hit Sequence 406.

When lines connecting all of the Hit Sequence endpoints are drawn to the Query Sequence 422 a series of Boundary Regions (hereinafter referred to as Regions) are visualized. A Region represents the sequence between two consecutive dashed lines connecting Hit Sequence endpoints to other Hit Sequences and the Query Sequence 422. Each Region (R₁ through R₅ in FIG. 4B) is identified and annotated to match the nucleotide sequence that it intersects in the initial Query Sequence 422 so that it can be related directly to a physical location on the original Query Sequence 422.

E. Alignment of Several Missing Nucleotides in a Hit Sequence with the Query Sequence.

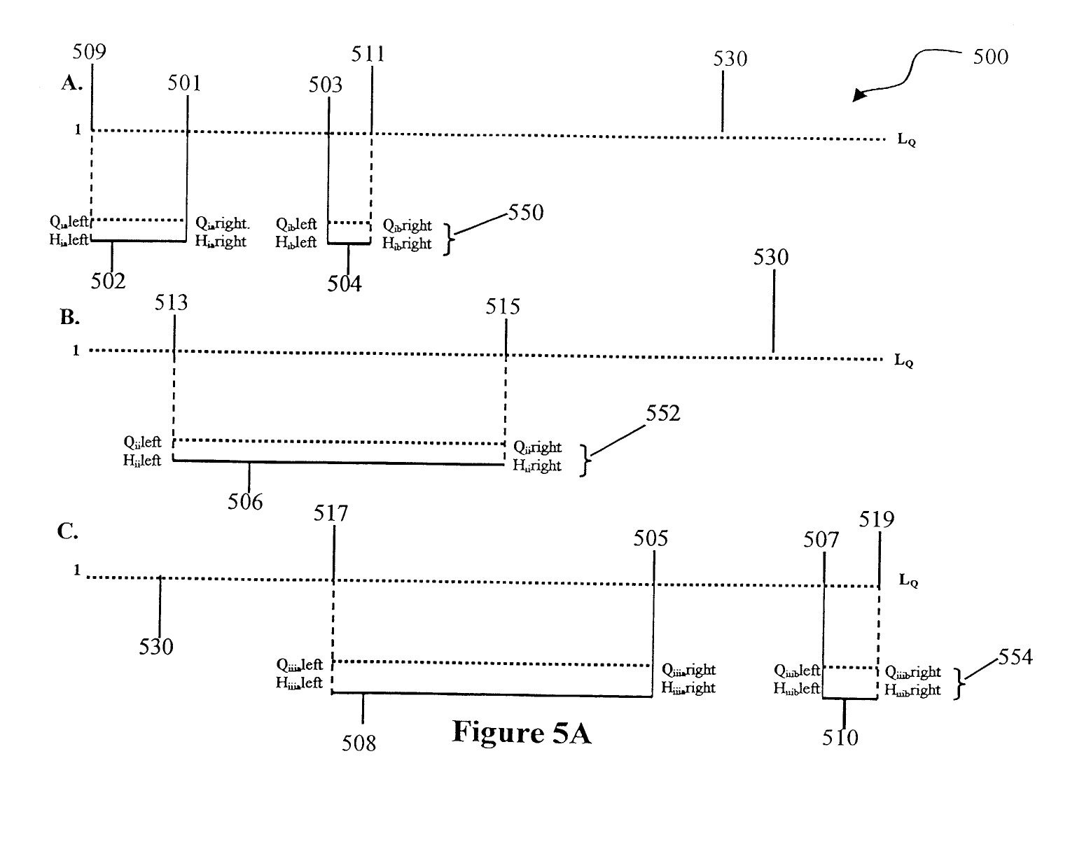

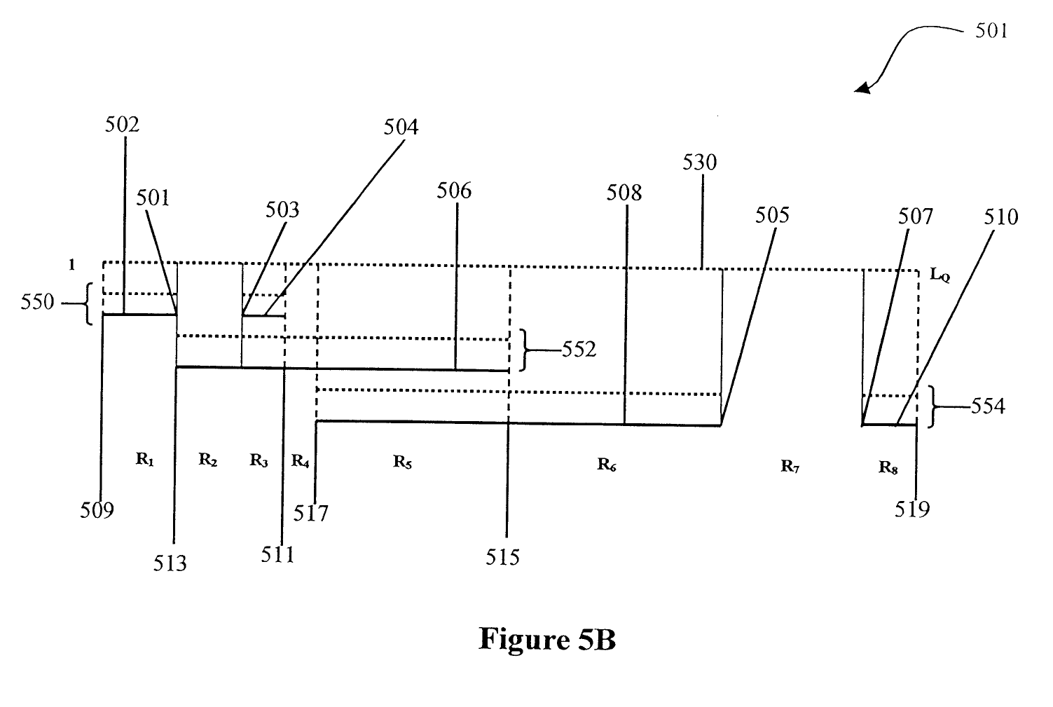

Any process for relating a plurality of Hit Sequences to a Query Sequence must take into account areas having several contiguous nucleotides that may be missing within the aligned Hit Sequence. FIG. 5A illustrates the relationship between Hit Sequences 502/504, 506 and 508/510 and the Query Sequence 530 where Hit Sequences 502/504 and 508/510 contain large open areas that are missing contiguous nucleotides, such areas having about 30 nucleotides or more, when aligned to the Query Sequence 530. These open areas arise during an alignment when there is not a homologous or similar sequence in the database Hit Sequence in relationship to the initial Query sequence 530. It may indicate that a fragment of that gene has been spliced out.

The first pairwise alignment 550 is composed of Hit Sequence 502/504 matching a portion of the Query Sequence 530 between points 509 and 511. The second pairwise alignment 552 is composed of Hit Sequence 506 matching a portion of the Query Sequence 530 between points 513 and 515. The third pairwise alignment 554 is composed of Hit Sequence 508/510 matching a portion of the Query Sequence 530 between points 517 and 519.

Defining Regions in Hit Sequences containing large open areas that are missing continuous nucleotides requires consideration of those open areas when defining Regions. In the presence of these open areas, lines are drawn from the endpoints of the open areas as well as the endpoints of the Hit Sequences. For example, in Hit Sequence 502/504 (shown in FIG. 5B) four lines are drawn that connect endpoints back to the Query Sequence 530. Dashed line 509 connects the left endpoint of the Hit Sequence 502 to the Query Sequence 530; solid line 501 connects the left endpoint of the open area (Region 2, R₂) of Hit Sequence 502 to the Query Sequence 530. Solid line 503 connects the right endpoint of the open area (Region 2, R₂) of Hit Sequence 504 to the Query Sequence 530 and dashed line 511 connects the right endpoint of the Hit Sequence 504 to the Query Sequence 530.

In Hit Sequence 506, solid line 513 and dashed line 515 are drawn from the left and right endpoints of that Hit Sequence 506 to the Query Sequence 530 respectively. The left endpoint of Hit Sequence 506 is a solid line because it overlays the left endpoint 501 of the open area of the Hit Sequence 504. Hit Sequence 508/510, contains an open area (Region 7, R₇) like Hit Sequence 502/504, and has a dashed line 517 connecting the left endpoint of the Hit Sequence 508 to the Query Sequence 530, solid line 505 connecting the left endpoint of its open area (Region 7, R₇) to the Query Sequence 530, solid line 507 connecting the right endpoint of the open area (Region 7, R₇) to the Query Sequence 530, and dashed line 519 connecting the right endpoint of Hit Sequence 510 to the Query Sequence 530.

Endpoint delineation of the Hit Sequences, including any open areas of about 30 nucleotides in length contained therein, is performed with lines drawn back to the Query Sequence 530. This process visualizes the Regions (R₁ through R₈). Each Region is defined on its right and left extremities by an endpoint line.

Whenever a defined Region represents a very small number of nucleotides, as for example less than about 5-10 nucleotides, those Regions can be ignored as an independent Region and incorporated into the next Region to prevent dilution of the significance of the delineated Regions.

### EXAMPLE 2

Unique Sequence Identification

Once the Regions have been defined for all Hit Sequences, the sequences, sequence fragments or open areas that are encompassed within each Region are determined. As illustrated in FIG. 4B, Region 1 (R₁) encompasses 1 matching sequence between end points 408 to 412; Region 2 (R₂) encompasses 2 matching sequence fragments between end points 412 to 410, R₃ encompasses 1 matching sequence fragment between end points 410 to 416; R₄ encompasses 2 sequence fragments between end points 416 to 414; and R₅ encompasses 1 matching sequence fragment between end points 414 to 418. There is at least one Hit Sequence in every Region on FIG. 4B therefore there are no Unique Sequences found on this Query Sequence.

As illustrated in FIG. 5B, Region 1 (R₁) encompasses 1 matching sequence fragment between end points 509 to 513, Region 2 (R₂) has one large open area with several missing nucleotides and 1 matching sequence fragment between end points 501/513 to 503. Region 3 (R₃) has 2 matching sequence fragments between end points 503 to 511 and R4 has 1 matching sequence fragment between end points 511 to 517. Region 5 (R₅) has 2 matching sequence fragments between 517 and 515 and R₆ has 1 matching sequence fragment between end points 515 to 505. Region 7 (R₇) has a large open area and has 0 Hit Sequences between end points 505 to 507 which match with the original Query. The last Region, R₈ has 1 matching sequence fragment between end points 507 to 519.

Region 2 between end points 501/513 to 503 and Region 7 between end points 505 to 507 both have open areas that have several contiguous missing nucleotides. Region 2 also has a Hit Sequence between end points 501/513 to 503 encompassed by that Region and therefore the area on the original Query which matches Region 2 is not defined as a Unique Sequence. Only the area on the original Query that matched 0 Hit Sequences, i.e. Region 7 between end points 505 to 507, is a Unique Sequence. Therefore the sequence fragment on the original Query between end points 505 and 507 encompassed within Region 7 is placed in the UNIQUE FILE.

If a Unique Sequence is identified as having fewer than 100 nucleotides, where 100 nucleotides is taken as the Expectation Threshold (ET), then it is disregarded. The ET is defined to be the length of a sequence alignment determined to be necessary to distinguish between evolutionary relationships and chance sequence similarity. Any sequence having fewer nucleotides than the selected ET is disregarded as a chance sequence similarity. However, the minimal ET selected may vary. For example, when a Hit contains an open area of unmatched nucleotides an ET of about 30 nucleotides may be selected.

### EXAMPLE 3

Building a Unique File

When first constructing the UNIQUE FILE 109 and 218 in FIG. 1B and 2 respectively, the file does not contain any Unique Sequences. A Unique Sequence is a sequence or sequence fragment on a Query Sequence 104 that is determined to be unique because it was never Hit or matched with a sequence from within the REP FILE or the UNIQUE FILE. Initially each Query Sequence 104 chosen from the subsets of RED FILES 102 in FIG. 1A is perceived as being a Unique Sequence excluding any Repeat Sequences that are identified during testing 105 and 214 in FIG. 1B and 2 respectively. This is because initially there are none or very few sequences that have been placed in the UNIQUE FILE 109 and 218 in FIG. 1B and 2 respectively. As the number of sequences or sequence fragments increase within the UNIQUE FILE 109 and 218 in FIG. 1B and 2 respectively, the likelihood of a Hit occurring on the Query Sequence 104 gets greater and fewer sequences or sequence fragments are placed in the UNIQUE FILE 109 and 218 in FIGS. 1B and 2 respectively.

### EXAMPLE 4

Using the Unique File

By using the Region Definition and Unique Sequence Identification Procedures (Examples 1 and 2) only one copy of any Query Sequence or sequence fragment can be placed within the UNIQUE FILE 109 and 218 in FIGS. 1B and 2 respectively. This is advantageous because the UNIQUE FILE 109 and 218 in FIGS. 1B and 2 respectively is then smaller and therefore much faster to test than any RED FILE subset 102, which contains Repeated Sequences.

Query Sequences 104 in FIG. 1A or Independently Derived sequences (ID) 212 in FIG. 2 can then be tested against the REP FILE 216 to eliminate Repeat Sequences and the UNIQUE FILE 218 to identify Unique Sequences faster and with more confidence than with any other method available.

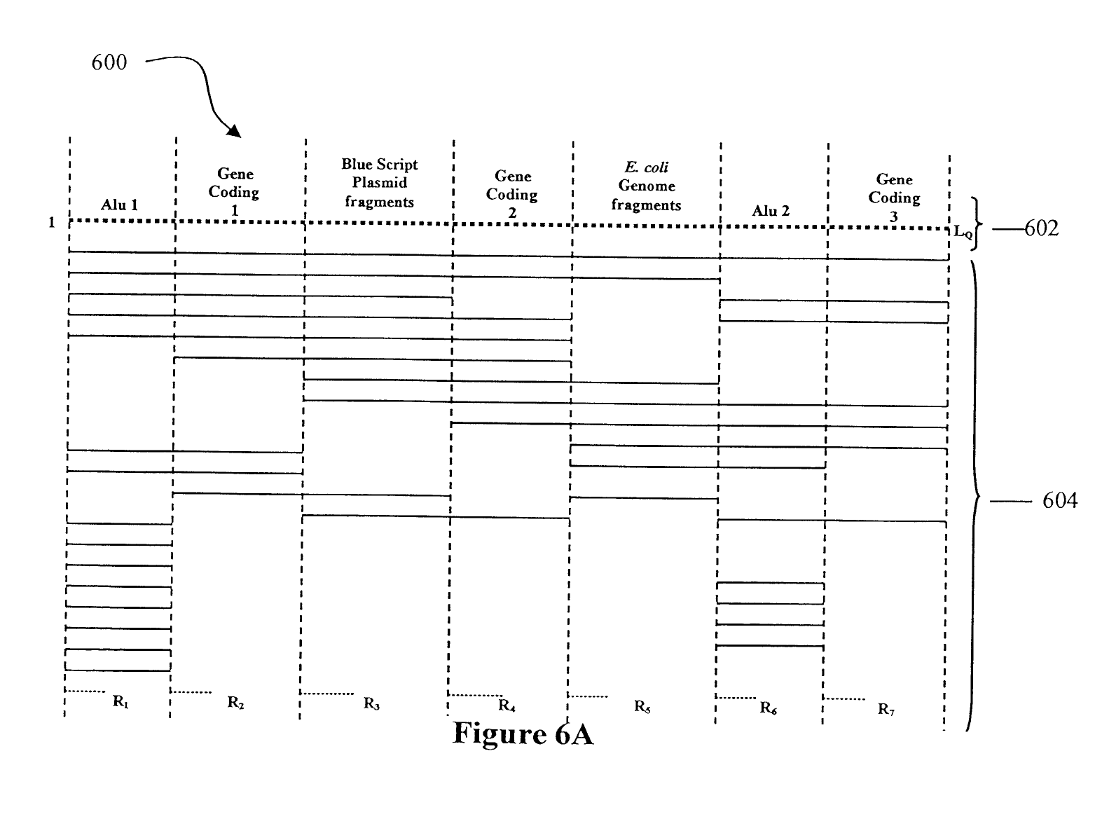

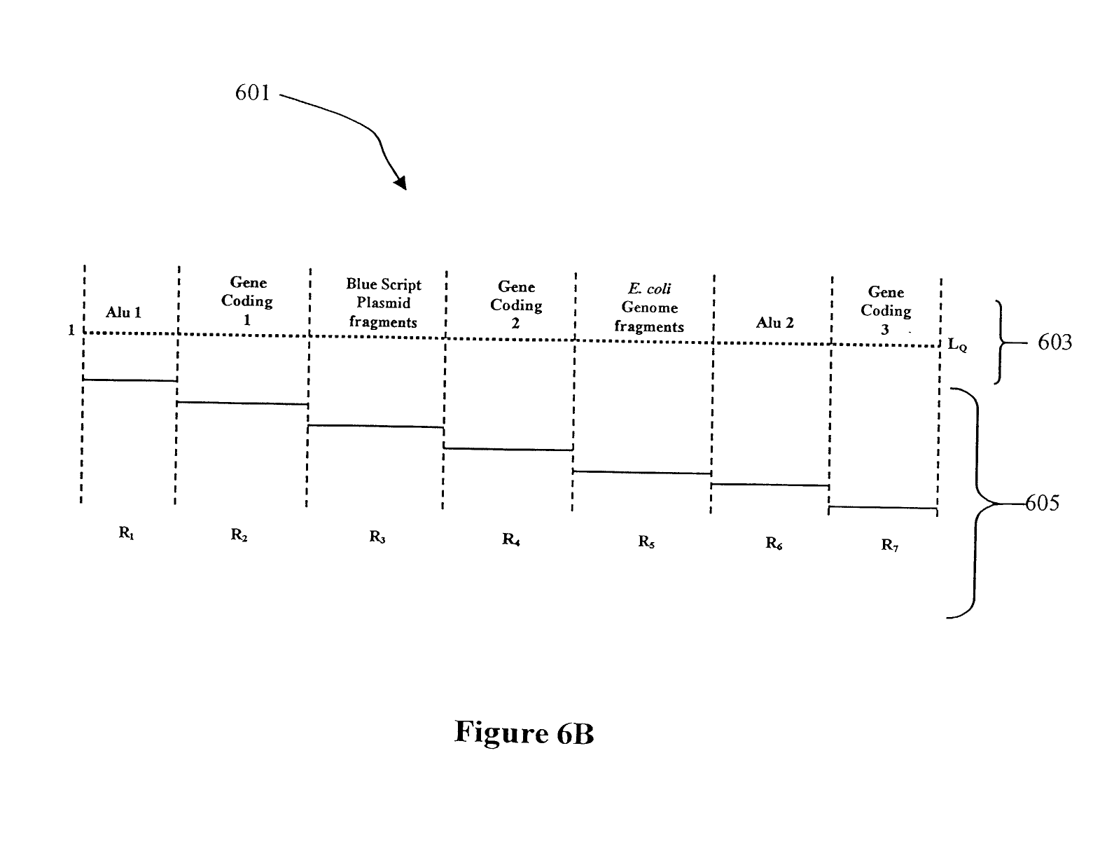

As shown in FIG. 6A when Query Sequence 602, containing contamination from Alu sequence fragments, Blue Script Plasmid sequence fragments, E. coli Genome sequence fragments along with the genes of interest, is searched against a RED BASE file multiple Hits 604 per Region Sequence or sequence fragments may be obtained. A Blast as shown in FIG. 6A using such a Query Sequence 602 against a RED BASE file may take as long as one hour to complete for a complicated, contaminated Query Sequence and may result in 10⁵ Hits or more.

In contrast, the UNIQUE FILE of the present invention provides a faster and more efficient use of resources. As illustrated in FIG. 6B when the Query Sequence 603 is searched against the UNIQUE FILE and the REP BASE file a maximum of one Hit 605 per Region Sequence or sequence fragment is obtained. A Query Sequence Blast as shown in FIG. 6B against the UNIQUE and REP BASE files will take 1 second and will result in no more than 1 Hit per Region, or a total of 7 or less Hits 605.

### REFERENCES

Altschul, Stephen F., Gish, W., Miller, W., Myers, W. W. and Lipman, David J. (1990). Basic Local Alignment Search Tool. J Mol. Biol. 215:403-410.

Altschul, Stephen F., Madden, Thomas L., Schaffer, Alejandro A., Zhang, Jinghui, Zhang, Zheng, Webb Miller, and Lipman, David J. (1997). Gapped BLAST and PSI-BLAST: a new generation of protein database search programs, Nucleic Acids Res. 25:3389-3402.

Dayhoff, M. O. (1978.), in Atlas of Protein Sequence and Structure, Vol 5, Suppl. 3, 229-249, National Biomedical Research Foundation, Washington, D.C., M. O. Dayhoff, ed.

Feng D. F., Johnson, M. S. and Doolittle, R. F. (1984-85). Aligning amino acid sequences: comparison of commonly used methods. J Mol Evol. 21(2):112-25.

Henikoff S., and Henikoff, J. G. (1992). Amino acid substitution matrices from protein blocks. Proc Natl Acad Sci U S A. November 15; 89(22):10915-9.

Karlin, S. and Ghandour, G. (1985). Multiple-alphabet amino acid sequence comparisons of the immunoglobulin kappa-chain constant domain. Proc Natl Acad Sci U S A. December; 82(24):8597-601.

Lipman, David J. and Pearson, W. R. (1985). Rapid and sensitive similarity searches. Science 227:1435-1441.

Pearson, W. and Lipman, David (1988). Improved tools for biological sequence comparison. Proc. Natl. Acad. Sci. 85:2444-2448.

Pearson, W. (1990). Rapid and sensitive sequence comparison with FASTP and FASTA. in Methods in Enzymology 183, Doolittle, R. ed. cf. pp. 75-85.

Smith, T. F. and Waterman, M. S. (1981). Identification of common molecular subsequences. J Mol. Biol. 147; 195-197.

Waterman, M. S. (1989). Sequence Alignments in Mathematical Methods for DNA Sequences, Waterman, M. S. ed. pp. 53-92. CRC Press, Boca Raton.

Waterman, M. S. (1995). Dynamic Programming Alignment of Two Sequences, in Introduction to Computational Biology: Maps, Sequences and Genomes. pp. 183-232, Chapman and Hall, New York.

All patents and publications mentioned in this specification are indicative of the level of skill of those of knowledge in the art to which the invention pertains. All patents and publications referred to in this application are incorporated herein by reference to the same extent as if each was specifically indicated as being incorporated by reference and to the extent that they provide materials and methods not specifically shown.

What is claimed is:

1. A database of unique nucleotide sequences, said database comprising nucleotide sequences greater than about 100 nucleotides in length.

2. A database of unique nucleotide sequences, said database comprising nucleotide sequences between about 100-500 nucleotides in length.

3. A database of unique nucleotide sequences, said database comprising nucleotide sequences between about 100-1000 nucleotides in length.

4. The database of any of claims 1-3, wherein said nucleotide sequence is a deoxyribonucleotide sequence.

5. The database of any of claims 1-3, wherein said nucleotide sequence is a ribonucleotide sequence.

6. The database of any of claims 1-3, wherein said sequences are derived from animal DNA or RNA.

7. The database of claim 6, wherein said animal is a human.

8. The database of claim 6, wherein said animal is a mouse.

9. The database of any of claims 1-3, wherein said sequences are derived from plant DNA or RNA.

10. The database of any of claims 1-3, wherein said plant is a single-cell plant.

11. The database of any of claims 1-3, wherein said sequences are derived from fungal DNA or RNA.

12. The database of any of claims 1-3, wherein said sequences are derived from DNA or RNA of a microorganism or virus.

13. The database of any of claims 1-3, wherein said sequences are derived from DNA or RNA of a single-cell eukaryote.

14. The database of any of claims 1-3, wherein said sequences are derived from synthetic man-made DNA or RNA.

15. The database of any of claims 1-3, wherein said sequences are postulated based upon amino acid sequences.

16. The database of any of claims 1-3, wherein said database is encoded in a biological medium.

17. The database of any of claims 1-3, wherein said database is encoded in a written medium.

18. The database of any of claims 1-3, wherein said database is encoded in an electronic medium.

19. The database of claim 18, wherein said electronic medium is a computer-readable medium.

20. The database of claim 19, wherein said computer-readable medium is addressable through an internet connection.

21. A kit for analyzing nucleotide sequences comprising:
an electronic medium readable by a computer, said medium encoding a database of unique nucleotide sequences, said database comprising nucleotide sequences greater than about 100 nucleotides in length.

22. A kit for analyzing nucleotide sequences comprising:
an electronic medium readable by a computer, said medium encoding a database of unique nucleotide sequences, said database comprising nucleotide sequences greater than about 100 nucleotides in length; and,
instructions for the use of said database.

23. A kit for analyzing nucleotide sequences comprising:
an electronic medium readable by a computer, said medium encoding a database of unique nucleotide sequences, said database comprising nucleotide sequences greater than about 100 nucleotides in length;
instructions for the use of said database; and,
a computer.

24. An improved database of nucleotide sequences, said database comprising nucleotide sequences greater than about 100 nucleotides in length, wherein said improvement consists entirely of only unique nucleotide sequences entered into said database only one time.

25. A computer-generated database consisting of only unique nucleotide sequences, said database comprising nucleotide sequences greater than about 100 nucleotides in length.

26. A method for generating a database of sequences that are greater than or equal to about 100 nucleotides in length, wherein each sequence is entered into the database only one time, the method comprising the steps of:
selecting a query sequence from a redundant database;
masking said query sequence with known repeat sequences;
comparing said masked query sequence with identified unique sequences;
identifying a unique portion of the query sequence that does not have a similar sequence in any of the identified unique sequences; and
adding the unique portion of the query sequence to a unique database.

27. A database product of the process of claim 26.

28. The method of claim 26, wherein said sequence is a deoxyribonucleotide sequence.

29. The method of claim 26, wherein said sequence is a ribonucleotide sequence.

30. The method of claim 26, wherein said sequences are derived from animal DNA or RNA.

31. The method of claim 30, wherein said animal is a human.

32. The method of claim 30, wherein said animal is a mouse.

33. The method of claim 26, wherein said sequences are derived from plant DNA or RNA.

34. The method of claim 33, wherein said plant is a single-cell plant.

35. The method of claim 26, wherein said sequences are derived from fungal DNA or RNA.

36. The method of claim 26, wherein said sequences are derived from DNA or RNA of a microorganism or virus.

37. The method of claim 26, wherein said sequences are derived from DNA or RNA of a single-cell eukaryote.

38. The method of claim 26, wherein said sequences are derived from synthetic man-made DNA or RNA.

39. The method of claim 26, wherein said sequences are postulated based upon amino acid sequences.

40. The method of claim 26, wherein said database is encoded in a biological medium.

41. The method of claim 26, wherein said database is encoded in a written medium.

42. The method of claim 26, wherein said database is encoded in an electronic medium.

43. The method of claim 42, wherein said electronic medium is a computer-readable medium.

44. The method of claim 43, wherein said computer-readable medium is addressable through an internet connection.

45. The method of claim 26, wherein said redundant database is a Public Domain Database.

46. The method of claim 45, wherein said Public Domain Database is GenBank.

47. The method of claim 45, wherein said Public Domain Database is dbEST.

48. The method of claim 45, wherein said Public Domain Database is TIGR.

49. The method of claim 45, wherein said Public Domain Database is SwissProt.

50. The method of claim 26, wherein said comparing step further utilizes a Database Search Algorithm.

51. The method of claim 50, wherein said Database Search Algorithm is BLAST.

52. The method of claim 50, wherein said Database Search Algorithm is FASTA.

53. The method of claim 50, wherein said Database Search Algorithm is Smith-Waterman.

54. The method of claim 26, wherein said comparing step further utilizes a Scoring Matrix Program.

55. The method of claim 54, wherein said Scoring Matrix Program is PAM.

56. The method of claim 54, wherein said Scoring Matrix Program is BLOSUM.

57. The process of FIG. 1A.

58. The process of FIG. 1B.

59. The process of FIG. 2.

60. A method for identifying unique nucleotide sequences, the method comprising the steps of:
selecting a query sequence from a redundant database file;
comparing the query sequence with a repeat database file and a unique database file;
analyzing the results of the comparison of the query sequence with the repeat database file and the unique database file to determine if there is one or more nucleotide sequences within the repeat database file and the unique database file that match a nucleotide sequence within the query sequence; and
identifying any unique nucleotide sequences within the query sequences that do not match any nucleotide sequence within the repeat database file and the unique database file.
